(19) United States
(12) Patent Application Publication
(10) Pub. No.: US 2006/0081255 A1
(43) Pub. Date: Apr. 20, 2006

(54) Ultrasonic placement and monitoring of an endotracheal tube

(76) Inventors: Michael Miller, Winnipeg (CA); Claire T. Hovland, Andover, MN (US)

Correspondence Address: CROMPTON, SEAGER & TUFTE, LLC, 1221 NICOLLET AVENUE, SUITE 800, MINNEAPOLIS, MN, 55403-2420, US

(21) Appl. No.: 11/094,849

(22) Filed: Mar. 31, 2005

Related U.S. Application Data
(60) Provisional application No. 60/559,325, filed on Apr. 2, 2004.

(51) Int. Cl. A62B 9/06; A61M 16/00

(52) U.S. Cl. 128/207.14; 128/207.15; 128/207.16

(57) ABSTRACT

A system for ultrasonically placing and monitoring an endotracheal tube within a patient. The system includes an endotracheal tube having a proximal and a distal end and a ventilation lumen disposed there through. A vibration mechanism is coupled to the endotracheal tube. One ultrasonic transducer is located outside the patient's body. An ultrasonic imaging apparatus is coupled to the ultrasonic transducer for digitalizing the endotracheal tube within the body.

This application claims the benefit of U.S. Provisional Application No. 60/559,325 as filed on Apr. 2, 2004.

### FIELD OF THE INVENTION

The present invention relates generally to the field of medical devices. More specifically, the present invention pertains to systems and methods for ultrasonic placement and monitoring of an endotracheal tube within the body.

### BACKGROUND OF THE INVENTION

A number of medical procedures require the insertion of a tube, catheter, cannula, or other similar device into the body. Such devices are used, for example, in the fields of anesthesiology, cardiology, endoscopy, urology, laparoscopy, and vascular therapy to deliver fluids such as oxygen and anesthetics to targeted regions within the body. In the field of anesthesiology and critical care, for example, it may be necessary to deliver air/oxygen to the anesthetized patient using an endotracheal tube (ETT). Such tubes are routine used in the clinical, ICU, emergency room, and pre-hospital settings to restore and maintain an adequate airway to the lungs, to prevent the inspiration of forced air into the stomach via the esophagus tube, and to protect against the aspiration of gastric contents into the lungs.

In a typical endotracheal intubation procedure, the distal end of the ETT is inserted through either the mouth or nose and is advanced into the trachea, generally at a location midway between the vocal folds and the carina. An inflatable balloon cuff located at or near the distal end of the ETT can be inflated to secure the ETT within the trachea, providing and air seal that allows the caregiver to completely control the flow of air provided to the lungs using an external ventilation unit, and that can be used to prevent the aspiration of gastric contents into the lungs.

The placement and monitoring of the ETT within the body remains a significant obstacle in endotracheal intubation procedures. Malpositioning may result when the ETT is inadvertently placed into the esophagus tube, causing air to be injected into the stomach instead of the trachea. Endobronchial intubation caused by over-extending the ETT past the carina and into one of the right or left primary bronchi may also exacerbate the intubation process, resulting in the ventilation of only one of the lungs. In certain circumstances, the lung that is being improperly ventilated may become hyperventilated due to the higher concentrations of inspired oxygen, causing barotraumas and hypotension. Atelectasis of the unventilated lung may also result from the improper insertion of the ETT into the bronchi.

Movement of the ETT once placed within the trachea may further exacerbate the intubation process. Flexion or extension of the patient's neck can change the desired positioning of the ETT, in some cases resulting in extubation from the trachea. Such changes in head position are common with normal patient movement in the ICU, emergency room, and pre-hospital settings. In addition, mucus, blood, or other biological materials may also result in the movement or blockage of the ETT, requiring further action by the caregiver to ensure proper ventilation of the patient. In any of these scenarios, the lack of proper ventilation within the patient may lead to cardiac arrest or irreversible central nervous system damage within a relatively short period of time.

The efficacy of endotracheal intubation procedure depends in part on the ability of the caregiver to quickly and accurately determine the positioning of the ETT within the body. Most intubation devices and methods rely on the ability to visualize the opening to the trachea and place the ETT by direct vision, typically with the aid of another instrument such as a fiber optic laryngoscope. Anatomical variations from patient to patient can, however, render direct visualization of the trachea opening difficult and in some cases impossible. This is particularly so during critical care and emergency procedures where the positioning of the patient's head or the presence of blood or saliva may exacerbate direct visualization. Post placement movement or blockage of the ETT may also be undetectable using direct visualization techniques, rendering this method ineffectual for monitoring of the ETT once inserted into the trachea.

To address these problems, various devices and techniques have been developed to aid in the proper placement and monitoring of the ETT within the body. Known techniques include, for example, chest radiography, stethoscopic evaluation of airway breath and epigastric sounds, visualization of the trachea and carina using a fiber optic bronchoscope, visualization of the vocal cords or trachea by video methods, pulse oximetry, carbon dioxide (CO₂) measurements, calorimetric end tidal CO₂ (ETCO₂) measurements, electromagnetic sensing, suction techniques, and the observation of symmetric bilateral movements of the chest wall during ventilation. A review of the various types of instruments utilized in the art is provided in U.S. Pat. No. 5,785,051 to Lipscher et al., which is incorporated herein by reference in its entirety.

More recent designs in the art have focused on ultrasonic techniques to monitor the placement of endotracheal tubes within the body. Such designs generally include an ultrasonic transducer mounted directly on the tube that can be used to transmit acoustic waves to a receiver located either on another portion of the tubular member, or to an external receiver located outside of the patient's body. In several prior art designs, the ability to ultrasonically visualize the tube is often dependent on the distance between the transducer and receiver, rendering such techniques prone to error in those applications where the distance is great, or where acoustical obstructions such as bone or air are present. In endotracheal intubation procedures, for example, a weak or nonexistent signal received from the transducer may falsely indicate that an esophageal intubation has occurred, requiring the caregiver to remove the ETT from the patient's body and reattempt the intubation process. Moreover, air located in the trachea, larynx, pharynx, and esophagus may impair ultrasonic imaging of these structures, affecting the ability of the caregiver to assess whether any contraindications to tracheal intubation exist.

While several prior art designs permit the caregiver to confirm the position of the tube once it has been placed in the body, such devices are not capable of ultrasonic placement and monitoring of the tube in real-time. Abnormalities in the airway and variations from patient to patient may render many ultrasonic techniques unsatisfactory for use. As such, there is a need in the art to provide real-time ultrasonic placement and monitoring of a tube within the body.

### BRIEF DESCRIPTION OF THE DRAWINGS

FIG. 1 is a diagrammatic view of an illustrative system for ultrasonically monitoring the placement of an endotracheal tube within the body;

FIG. 2 is a perspective view showing the illustrative endotracheal tube of FIG. 1 in greater detail;

FIG. 3 is an assembly view of an illustrative ventilation hub and L-shaped adapter;

FIG. 4 is an assembly view showing the attachment of an illustrative vibration mechanism to the ventilation hub and L-shaped adapter of FIG. 3;

FIG. 5 is a front view of an ultrasound apparatus in accordance with an illustrative embodiment of the present invention;

FIG. 6 is a side view of the ultrasound apparatus of FIG. 5;

FIG. 7 is a top view of the ultrasound apparatus along line 7-7 of FIG. 6;

FIG. 8 is a front view of an ultrasound apparatus in accordance with another illustrative embodiment of the present invention;

FIG. 9 is a front view of an ultrasound apparatus in accordance with another illustrative embodiment the present invention;

FIG. 10 a front view of an ultrasound apparatus in accordance with another illustrative embodiment of the present invention;

FIG. 11 is a first view showing the initial insertion of the endotracheal tube of FIG. 2 within the airway of a patient;

FIG. 12 is a second view showing the endotracheal tube in a second position at or near the epiglottis; and

FIG. 13 is a third view showing the endotracheal tube in a third position inflated within the patient's trachea.

### DETAILED DESCRIPTION OF THE INVENTION

The following description should be read with reference to the drawings, in which like elements in different drawings are numbered in like fashion. The drawings, which are not necessarily to scale, depict selected embodiments and are not intended to limit the scope of the invention. Although examples of construction, dimensions, and materials are illustrated for the various elements, those skilled in the art will recognize that many of the examples provided have suitable alternatives that may be utilized.

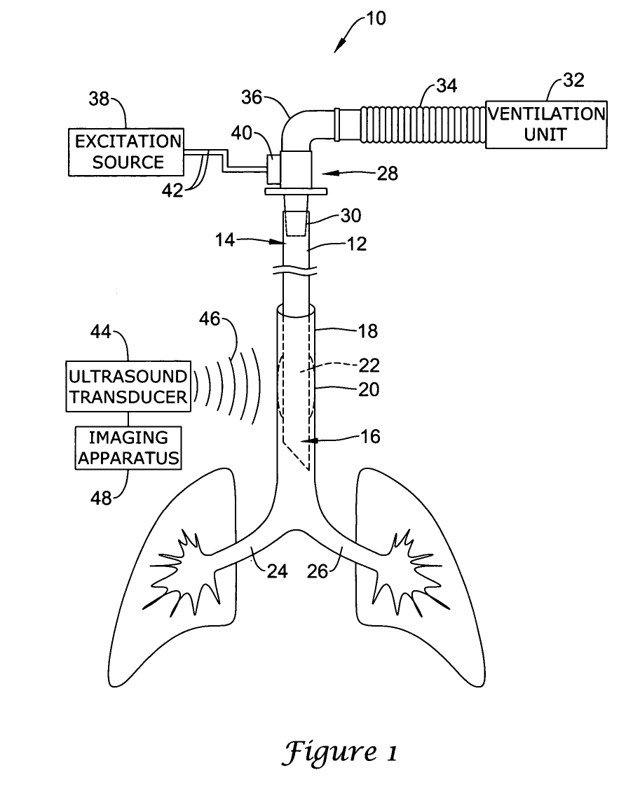

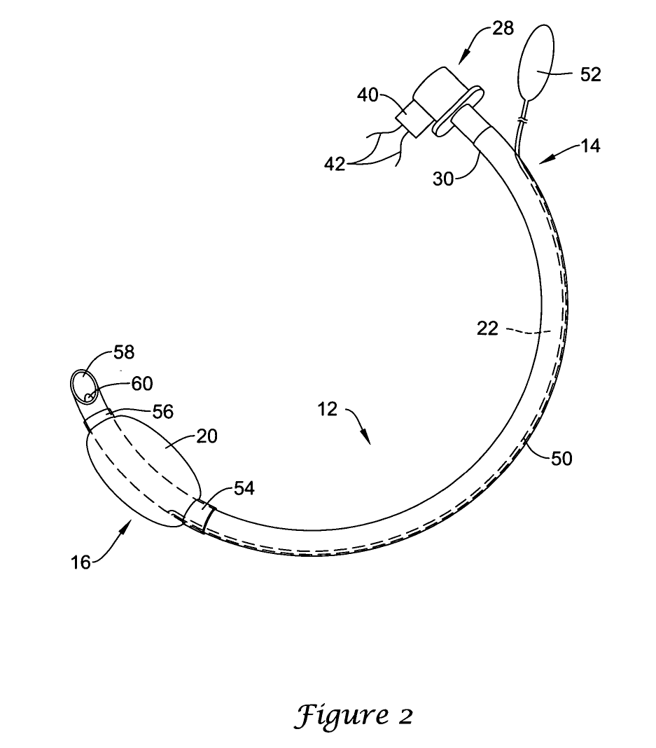

FIG. 1 is a diagrammatic view of an illustrative system 10 for ultrasonically monitoring the placement of an endotracheal tube (ETT) 12 within the body. As shown in FIG. 1, the endotracheal tube 12 can include a proximal section 14 that can be manipulated from a position outside of a patient's body during the intubation procedure, and a distal section 16 that can be advanced within the patient's airway to a desired location within the trachea 18. As is discussed in greater detail with respect to FIG. 2, the endotracheal tube 12 can include an inflatable cuff 20 that can be expanded to secure the endotracheal tube 12 to the interior wall of the trachea during intubation. A ventilation lumen 22 of the endotracheal tube 12 can be used to provide air, anesthetics, or other vital fluids the patient's right and left bronchi 24,26.

A ventilation hub 28 coupled to a proximal end 30 of the endotracheal tube 12 can be utilized to fluidly couple the ventilation lumen 22 of the endotracheal tube 12 to an external ventilation unit 32 that can be used for ventilating the patient, and for delivering anesthetics, antibiotics and other drugs to the patient. A ventilation hose 34 having one or more lumens therein can be used to deliver and receive fluids to and from the endotracheal tube 12. The ventilation hose 34 can be releasably connected to the ventilation hub 28 via an optional L-shaped adapter 36.

An excitation source 38 can be provided to vibrate the endotracheal tube 12, allowing the positioning and placement of the endotracheal tube 12 to be monitored in real-time from a position outside of the patient's body. A vibration mechanism 40 electrically coupled to the excitation source 38 via a number of electrical leads 42 can be configured to produce vibration at the ventilation hub 28, which is then transmitted into the attached endotracheal tube 12 and delivered to the distal section 16. The vibration mechanism 40 can be coupled to or formed integrally with a portion of the ventilation hub 28, as shown in FIG. 1, or can be attached directly to a portion of the endotracheal tube 12, if desired. In use, the excitation source 38 can be configured to provide a time-varying voltage signal to the vibration mechanism 40 to drive a speaker, piezoelectric actuator, motor, or other suitable vibration means.

An ultrasonic transducer 44 located outside of the patient's body can be utilized to ultrasonically monitor the location of the endotracheal tube 12 within the patient's airway. In certain embodiments, the ultrasonic transducer 44 can be configured to measure phase shifts in the frequency of an incident wave 46 caused by the reflection of the incident wave 46 against the vibrating endotracheal tube 12. As shown in FIG. 1, for example, an incident wave pulse 46 emitted from the ultrasonic transducer 44 ex vivo can be directed through the skin and into a target region within the body (e.g. the trachea, larynx/pharynx, vocal cords, etc.). When the incident wave pulse 46 is reflected against the vibrating endotracheal tube 12, a slight phase shift in the frequency will occur as a result of the vibrations, which can then be measured with the ultrasound transducer 44 using Doppler ultrasound techniques.

An ultrasound imaging apparatus 48 can be used to visualize the vibrating endotracheal tube 12 in real-time, if desired. In certain embodiments, for example, the ultrasound imaging apparatus 48 can include a color Doppler ultrasound monitor that can be used to distinguish between movement of the endotracheal tube 12 and the surrounding anatomy. The ultrasonic imaging apparatus 48 and ultrasound transducer 44 can be provided as a single, portable unit that can be used in a pre-hospital setting. Alternatively, the ultrasonic imaging apparatus 48 and ultrasound transducer 46 can be provided as separate units, if desired. While it is contemplated that ultrasonic imaging techniques could be used to ultrasonically monitor the position of the endotracheal tube 12 within the body, it should be understood that other devices could be utilized. In one alternative embodiment, for example, an auscultatory monitor (e.g. Doptone®) capable of producing an audible signal in response to Doppler movement of the endotracheal tube 12 could be employed.

FIG. 2 is a perspective view showing the illustrative endotracheal tube 12 of FIG. 1 in greater detail. As can be seen in FIG. 2, the endotracheal tube 12 can define an inflation lumen 50 that can be used to deliver fluid to the inflatable cuff 20 via an external fluid reservoir 52 such as an elastomeric bulb, syringe mechanism or the like. The inflatable cuff 20, which is secured to the outer surface of the endotracheal tube 12 via a number of cuffs 54,56, can be configured to inflate when fluid (e.g. air, saline solution, etc.) located in the external fluid reservoir 52 is injected into inflation lumen 50.

The distal section 16 of the endotracheal tube 12 may have a beveled shape, forming a tip 58 on the posterior wall of the endotracheal tube 12 that exposes the ventilation lumen 22 to the surrounding airway. The tip 58 may comprise a material that is sufficiently soft and flexible to prevent trauma to the body as the endotracheal tube 12 is advanced within the patient's body. In certain embodiments, a Murphy eye 60 located on the posterior wall of the endotracheal tube 12 may also be provided to prevent complete blockage of the endotracheal tube 12 in the event the tip 58 becomes partially or totally occluded.

The endotracheal tube 12 may comprise a suitably flexible material to permit it to be easily inserted into the patient's airway. The endotracheal tube 12 may also be provided with sufficient rigidity along its length to withstand buckling and transmit torque as it is inserted into the body. In certain embodiments, the endotracheal tube 12 may have a substantially curved shape along its length that approximates the contour of the patient's airway, allowing the device to follow a pre-guided path through the anterior portion of the larynx/pharynx and into the trachea. Other configurations such as a substantially straight shape may also be implemented, if desired.

The endotracheal tube 12 may have a length of approximately 9 to 15 inches and an outer diameter of about 0.7 cm to 1.1 cm, which is suitable for most adult orotracheal intubation procedures. The dimensions of the endotracheal tube 12 may, however vary for use in other applications, as necessary. In intubations for small infants, for example, the length and cross-sectional area of the endotracheal tube 12 can be scaled down to accommodate the relatively small size of the undeveloped infant trachea, which is typically about 4 cm in length and 0.5 cm in diameter. Moreover, where orotracheal intubation is unfeasible or contraindicated (e.g. in the case of a suspected cervical spine injury), the endotracheal tube 12 can be appropriately sized to permit alternative intubation techniques such as nasotracheal intubation or cricothyrotomy. The dimensions of the endotracheal tube 12 can also be altered to permit the device to be used in other fields such as veterinary medicine, if desired.

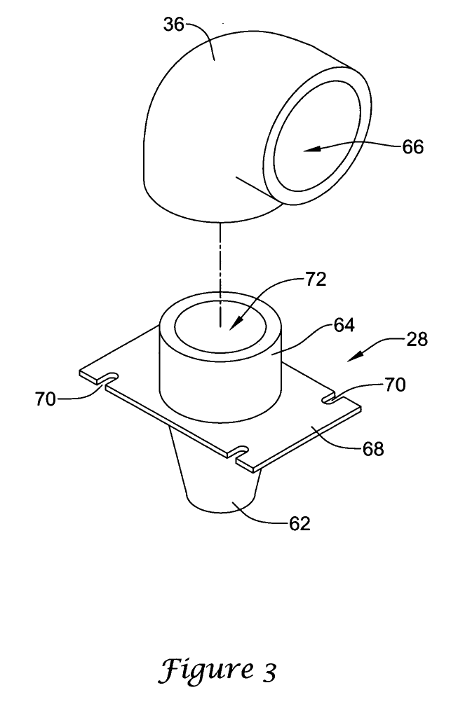

FIG. 3 is an assembly view showing the connection of the ventilation hub 28 to the L-shaped adapter 36. As can be seen in FIG. 3, the ventilation hub 28 can include a tapering nub 62 adapted to be push-fit tightly within ventilation lumen 22 of the endotracheal tube 12 (not shown), and a constant-diameter base 64 adapted to fit tightly within an interior lumen 66 of the L-shaped adapter 36. A flanged portion 68 of the ventilation hub 28 can be configured to act as a shoulder for the L-shaped adapter 36 when push-fit over the constant-diameter base 64. In certain embodiments, the flanged portion 68 can include a number of notches 70 that can be used to secure the ventilation hub 28 to an endotracheal tube holder or other the similar apparatus. When assembled, an internal lumen 72 extending through the ventilation hub 28 fluidly connects the interior lumen 66 of the L-shaped adapter 36 to the ventilation lumen 22 of the endotracheal tube 12.

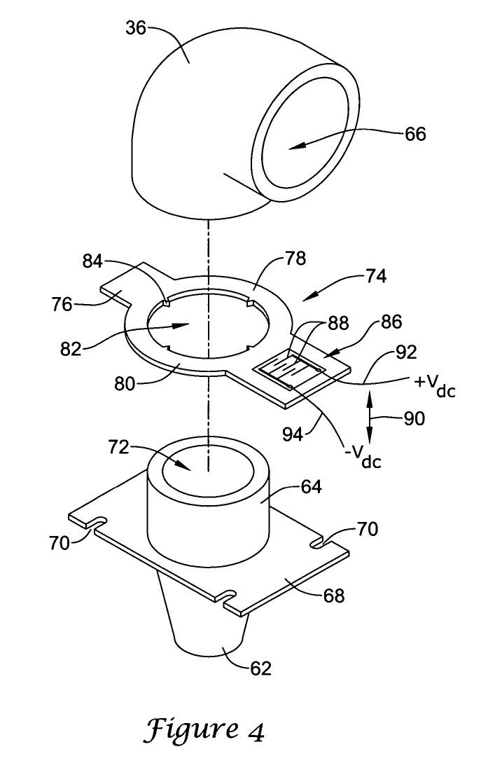

FIG. 4 is an assembly view showing the attachment of an illustrative vibration mechanism 74 to the ventilation hub 28 and L-shaped adapter 36 of FIG. 3. As shown in FIG. 4, the vibration mechanism 74 can include a thin plate 76 having an upper surface 78, a lower surface 80, and an opening 82 therethrough that can be dimensioned to tightly fit about the constant-diameter base 64 of the ventilation hub 28. A number of inwardly projecting teeth 84 can be configured to frictionally engage the constant-diameter base 64, providing a tight connection between the thin plate 76 and ventilation hub 28. When assembled, the lower surface 80 of the thin plate 76 can be configured to lie flush against the flanged portion 68 of the ventilation hub 28 in a manner similar to that of a washer, allowing the L-shaped adapter 36 to be push fit about the constant-diameter base 64 and secured thereto.

A vibration actuator 86 coupled to the upper and/or lower surfaces 78,80 of the vibration mechanism 74 can be activated to induce vibration in the adjacent ventilation hub 28, which can then be transmitted to the distal section 16 of the endotracheal tube 12. In the illustrative embodiment of FIG. 4, the vibration actuator 86 includes a Macro Fiber Piezocomposite (MFP) actuator having a number of interdigitated electrodes 88 that can be used to oscillate the MFP actuator in a direction indicated generally by reference arrow 90. A number of electrode leads 92,94 disposed on the MFP actuator 86 can be utilized to electrically couple the actuator 86 to a DC voltage source V_{DC} that can be used to drive the vibration actuator 86. The voltage drive source V_{DC} can be configured to output a time-varying voltage signal to alternate the charge delivered to the electrode leads 92,94, causing the vibration actuator 86 to oscillate back and forth. The vibration induced within the vibration mechanism 74 is then transmitted to the adjacent ventilation hub 28 and into the endotracheal tube 12, inducing a transverse-mode vibration along the entire length of the endotracheal tube 12 that can be used to ultrasonically monitor and visualize the precise location of the endotracheal tube 12 using Doppler ultrasound techniques.

The characteristics of the drive voltage V_{DC} signal applied to the vibration actuator 86 can be varied to alter the vibrational characteristics induced within the endotracheal tube 12. In certain embodiments, for example, the amplitude and frequency of the drive voltage V_{DC} can be adjusted to alter the vibration occurring along the length of the endotracheal tube 12. A drive voltage V_{DC} signal having a frequency within the range of 2 Hz to 2000 Hz, and more specifically 10 Hz to 200 Hz, and more specifically 15 Hz to 100 Hz, can be used to produce low-frequency vibrations within the endotracheal tube 12 that are generally inaudible to the human-hear. It should be understood, however, that frequencies above and below these ranges could be used to vibrate the endotracheal tube 12, if desired. As the vibration frequency increases beyond a certain rate (e.g. 1500 Hz), however, the ability to ultrasonically detect motion of the distal section 16 of the endotracheal tube 12 using Doppler ultrasound techniques diminishes.

While an MFP actuator 86 is specifically shown in the illustrative embodiment of FIG. 4, it should be understood that other vibration actuators could be employed. Other suitable vibration actuators that can be utilized in accordance with the present invention include, but are not limited to, an offset DC rotary motor, an AC solenoid, piezoelectric actuators (e.g. bimorph, stack actuators, ring actuators, etc.), a speaker (e.g. electrostatic, moving coil, etc.) or the like.

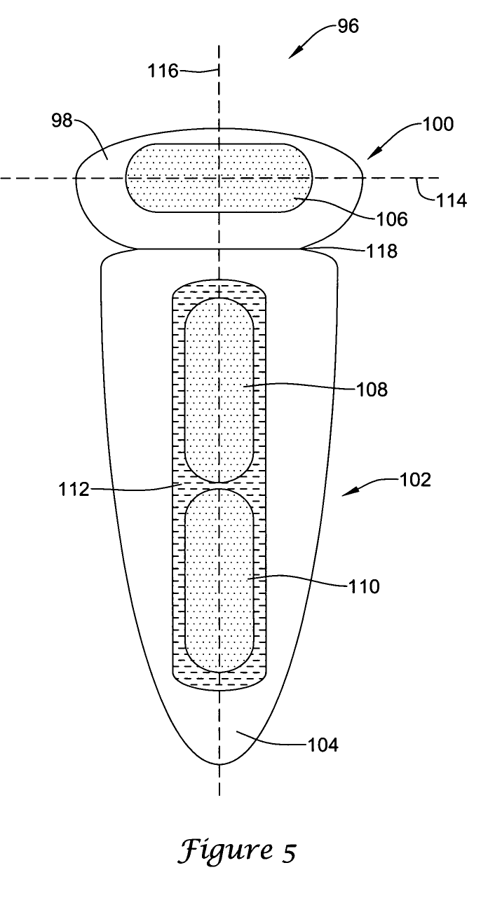

FIG. 5 is a front view of an ultrasound apparatus 96 in accordance with an illustrative embodiment of the present invention for ultrasonically visualizing the endotracheal tube 12. Ultrasound apparatus 96 can include a mandible 98 having an upper section 100 that can be positioned on the anterior surface of the patient's neck adjacent the upper (i.e. superior) end of the patient's airway, and a lower section 102 that can be positioned on the anterior portion of the patient's neck adjacent the lower (i.e. inferior) end of the patient's airway. The mandible 98 can be dimensioned to contour to the patient's body, having a relatively wide shape at the upper section 100 for positioning on the anterior surface of the neck, and a longer, narrower shape at the lower section 102 for positioning on the anterior surface of the sternum. A sternal notch 104 on the lower section 102 of the mandible 98 can be used for positioning the lower section 102 on the anterior surface of the sternum. In use, a neoprene rubber strap (not shown) or other suitable fastening means can be employed to secure the mandible 98 firmly against the patient's skin.

The mandible 98 can include a number of ultrasonic transducers for transmitting and receiving ultrasonic waves through the skin and into various locations within the patient's airway. A first ultrasonic transducer 106 located on the upper section 100 of the mandible 98 can be configured to transmit and receive ultrasonic waves to an upper portion of the patient's airway to monitor the placement of the endotracheal tube 12 as it is first instead into the mouth or nasal cavity and advanced to a position at or near the epiglottis. A second and third ultrasonic transducer 108,110, in turn, can be positioned on the lower section 102 of the mandible 98 for transmitting and receiving ultrasonic waves that can be used to monitor the endotracheal tube 12 as it is further inserted distally into the patient's airway. The second and third ultrasonic transducers 108,110 can be isolated from each other and the surrounding surface of the mandible 98 via a baffle layer 94 of foam, gel-pad, rubber, or other acoustically absorptive material. A similar absorptive baffle layer (not shown) may also be provided for the first ultrasonic transducer 106, if desired.

The ultrasonic transducers 106,108,110 can be oriented in various positions to focus and direct the ultrasonic waves to desired features within the body. The first ultrasonic transducer 106, for example, can include major length oriented along a horizontal axis 114, and a minor length oriented along a vertical axis 116. The second and third ultrasonic transducers 108,110, in turn, can each include a major length oriented along the vertical axis 116 substantially perpendicular to the first ultrasonic transducer 106. Each ultrasonic transducer 106,108,110 can include one or more ultrasonic transducer elements that can be selectively activated to ultrasonically monitor the location of the endotracheal tube 12 at various locations within the patient's airway. The particular shape of the ultrasonic transducer 106,108,110 can be configured to easily direct ultrasonic waves at key locations within the body, including, for example, the larynx, pharynx, trachea, vocal folds, epiglottis, and carina.

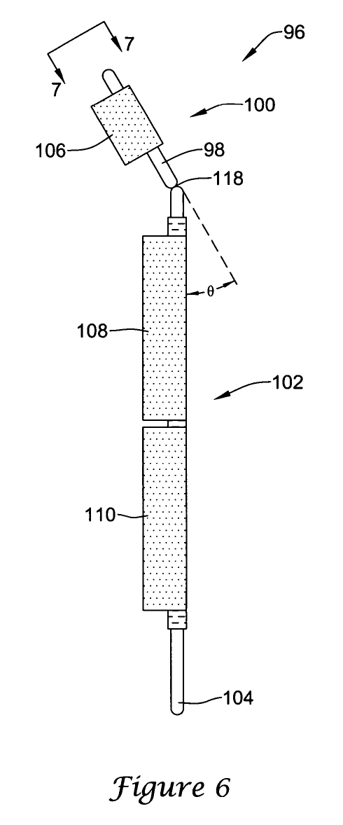

FIG. 6 is a side view of the ultrasound apparatus 96 of FIG. 5. As shown in FIG. 6, the mandible 98 can be configured to adjustable bend about a bendable joint 118, allowing the upper section 100 of the mandible 98 to bend at an angle θ relative to the lower section 102 of the mandible 98. In certain embodiments, the upper and lower sections 100,102 of the mandible 98 can be biased to assume a substantially straight position (i.e. θ=0°) until deflected by placement of the apparatus 96 on the anterior portion of the patient's neck.

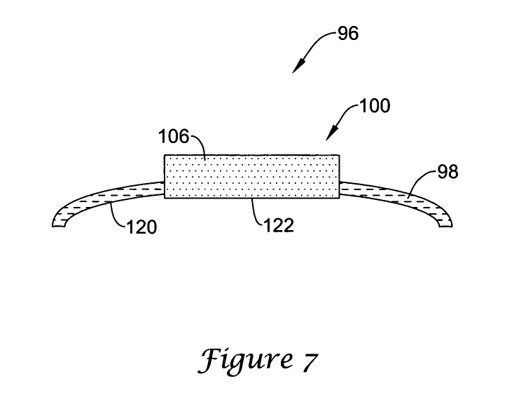

FIG. 7 is a top view of the ultrasound apparatus 96 along line 7-7 of FIG. 6. As can be seen in FIG. 7, the upper section 100 of the mandible 98 can have a concaved surface 120 that partially surrounds the anterior surface of the patient's neck to hold the first ultrasonic transducer 106 firmly thereto, when attached. This ensures that the leading surface 122 of the ultrasonic transducer 106 comes into close contact with the anterior skin surface of the patient's neck irrespective of the angle θ at which the upper section 100 is oriented with respect to the lower section 102.

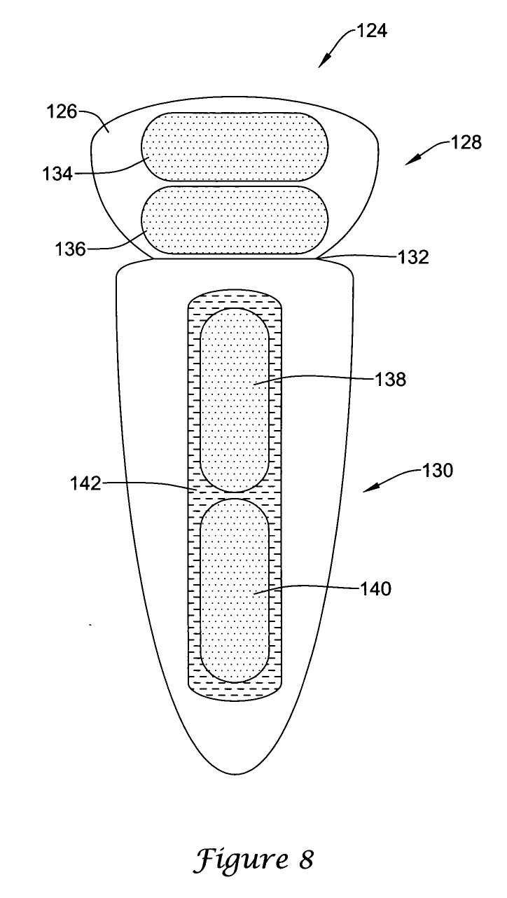

FIG. 8 is a front view of an ultrasound apparatus 124 in accordance with another illustrative embodiment of the present invention. Ultrasound apparatus 124 can include a mandible 126 having an upper section 128 that can be positioned on the anterior surface of the patient's neck at or near the upper end of the patient's airway, and a lower section 130 that can be positioned on the anterior portion of the patient's neck at or near the lower end of the patient's airway. A bendable joint 132 similar to that described above with respect to FIG. 6 can be employed to permit the upper section 128 to bend relative to the lower section 130, if desired.

A first and second ultrasonic transducer 134,136 disposed on the upper section 128 of the mandible 126 can be configured to transmit and receive ultrasonic waves to an upper portion of the patient's airway to monitor the placement of the endotracheal tube 12 as it is first instead into the mouth or nasal cavity and advanced to a position at or near the epiglottis. As with the first ultrasonic transducer 106 described above with respect to FIG. 5, the first and second ultrasonic transducers 134,136 can have a major length oriented in a substantially horizontal direction.

A third and fourth ultrasonic transducer 138,140 disposed on the lower section 130 of the mandible 126 can be utilized for transmitting and receiving ultrasonic waves for monitoring the endotracheal tube 12 as it is further inserted distally into the patient's airway. As with the previous embodiment, the second and third ultrasonic transducers 138,140 can be isolated from each other and the surrounding surface of the mandible 126 via a baffle layer 142.

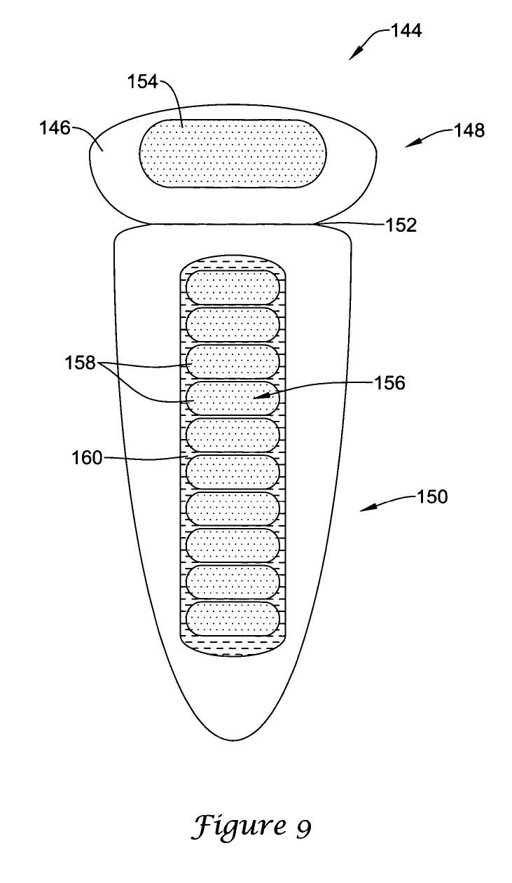

FIG. 9 is a front view of an ultrasound apparatus 144 in accordance with another illustrative embodiment of the present invention. Ultrasound apparatus 144 can include a mandible 146 having an upper section 148 that can be positioned on the anterior surface of the patient's neck adjacent the upper end of the patient's airway, and a lower section 150 that can be positioned on the anterior portion of the patient's neck adjacent the lower end of the patient's airway. A bendable joint 152 can be employed to permit the upper section 148 to bend relative to the lower section 150, if desired.

A first ultrasonic transducer 154 disposed on the upper section 148 of the mandible 146 can be configured to transmit and receive ultrasonic waves to an upper portion of the patient's airway. A vertical array 156 of ultrasonic transducers 158 each stacked vertically and in close proximity to each other can be used to transmit and receive ultrasonic waves for monitoring the endotracheal tube 12 as it is further inserted distally into the patient's airway. As with other embodiments described herein, each ultrasonic transducer 158 can be isolated from each other and the surrounding surface of the mandible 146 via a baffle layer 160.

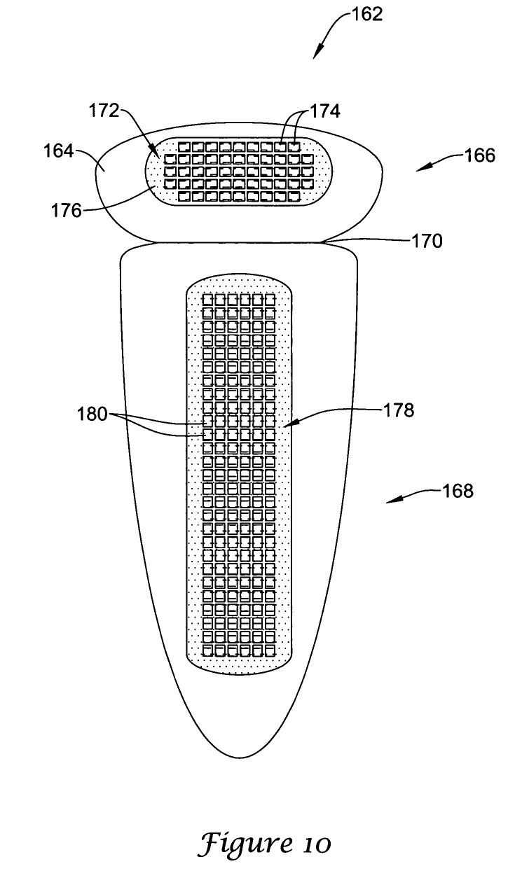

FIG. 10 a front view of an ultrasound transducer apparatus 162 in accordance with another illustrative embodiment of the present invention. Ultrasound apparatus 162 can include a mandible 164 having an upper section 166 that can be positioned on the anterior surface of the patient's neck adjacent the upper end of the patient's airway, and a lower section 168 that can be positioned on the anterior portion of the patient's neck adjacent the lower end of the patient's airway. A bendable joint 170 can be employed to permit the upper section 166 to bend relative to the lower section 168, if desired.

A first ultrasonic transducer 172 on the upper section 166 of the mandible 164 can include a number of individual ultrasonic transducer elements 174 that can be individually activated to transmit and receive one or more ultrasonic waves to an upper portion of the patient's airway. The ultrasonic transducer elements 174 can be arranged in a two-dimensional array having multiple horizontal ultrasonic transducer elements and vertical ultrasonic transducer elements. Each transducer element 174 within the transducer array can be isolated from each other and the surrounding surface of the mandible 164 via a baffle layer 176.

A second array 178 of ultrasonic transducer elements 180 disposed on the lower section 168 of the mandible 164 can be selectively activated to transmit and receive ultrasonic waves that can be used for monitoring the location of the endotracheal tube 12 as it is further inserted distally into the patient's airway. As with the first ultrasonic transducer 172, each of the individual ultrasonic transducer elements 180 can be arranged in a two-dimensional array having both a number of horizontal ultrasonic transducer elements and vertical ultrasonic transducer elements.

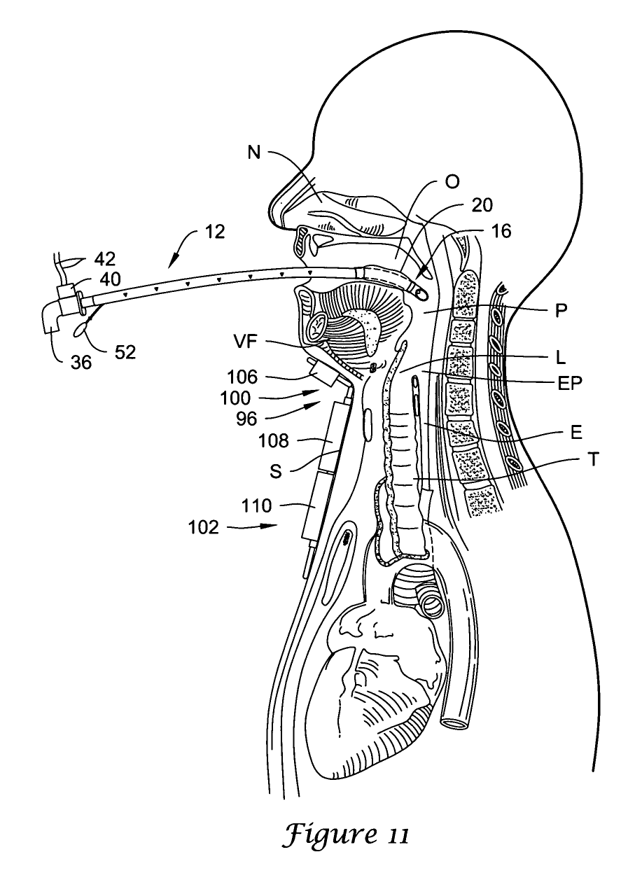

Referring now to FIGS. 11-13, an illustrative method of ultrasonically placing and monitoring an endotracheal tube within the body will now be described in the context of an orotracheal intubation procedure using the endotracheal tube 12, vibration mechanism 40, and ultrasound apparatus 96 described above. While specific reference is made to endotracheal intubation procedures, it should be understood that the methods described herein could be used in a number of other medical procedures to place and monitor tubes within the body. The methods described herein, for example may be used in vascular interventional procedures to place and monitor tubes used in vascular brachytherapy, angioplasty, stent placement, vascular catheter placement, or the like. Other medical fields including, for example, endoscopy, cardiology, urology, laparoscopy, obstetrics, neurology, radiology, and emergency medicine may also benefit from the methods described herein.

FIG. 11 is a cross-sectional view showing the initial insertion of the endotracheal tube 12 within the body. Prior to this point, and in preparation for the intubation procedure, the caregiver places the ultrasonic apparatus 96 about the anterior surface S of the patient's neck and sternum with the upper section 100 being positioned adjacent the upper end of the airway and the lower section 102 positioned adjacent the lower end of the airway. A gel material, gel pad, or other suitable acoustically transmissive material and/or structure can be placed between the contact surfaces of the ultrasonic transducers 106,108,110 and the anterior surface S of the skin to reduce reflection loss. An optional neck strap or other suitable fastening mechanism (not shown) can also be used to secure the ultrasonic apparatus 96 to the anterior surface S, if desired.

The ultrasonic apparatus 96 can be connected to an external ultrasonic monitor that can be used to visualize the larynx L, pharynx P, trachea T, vocal folds VF as well as other surrounding anatomy prior to insertion of the endotracheal tube 12 within the body. Such initial step may be performed, for example, to assess whether any abnormalities exist that may make the intubation process difficult, or in determining whether alternative airway management methods are indicated. In certain circumstances, for instance, an initial ultrasonic scan of the patient's airway may lead to the discovery of an obstruction in the upper portion of the trachea, indicating that an alternative method such as a cricothyrotomy may be necessary.

Ultrasonic imaging of the larynx L, pharynx P, vocal folds VF, trachea T, and surrounding anatomy can be accomplished using any number of suitable ultrasonic imaging techniques in the art, including, for example, A mode imaging, B mode imaging, C mode imaging, M mode imaging, Doppler or Duplex imaging, and/or Power Doppler imaging. In certain embodiments, the ultrasonic transducer and monitor may be provided as a single, portable unit that can be used in a pre-hospital setting such as at an accident site or in an ambulance. Such portable ultrasonic devices are commercially available from SonoSite, Inc. of Brothell, Wash.

Once the caregiver has determined that tracheal intubation is appropriate, a metal stylet or other stiffening member may be temporarily inserted into the ventilation lumen 22 of the endotracheal tube 12 to provide rigidity for the intubation process. With the ultrasonic apparatus 96 positioned on the patient's neck and sternum, the caregiver next activates the vibration mechanism 40 to vibrate the distal section 16 of the endotracheal tube 12.

With the vibration mechanism 40 activated, the caregiver next inserts the endotracheal tube 12 and accompanying metal stylet into the patient, either through the mouth or the nose in accordance with standard practice in the art. In an orotracheal intubation approach illustrated in FIG. 11, for example, the distal section 16 of the endotracheal tube 12 is shown inserted through the patient's oral cavity O, and then advanced to the region of the vocal folds VF. During this process, the inflatable cuff 20 can be maintained in a deflated position to facilitate passage of the endotracheal tube 12 through the airway.

While an orotracheal intubation approach is specifically shown in FIG. 11, it should be understood that the endotracheal tube 12 could also inserted through the patient's nasal cavity N if a nasotracheal intubation approach is indicated. In such approach, the distal section 16 of the endotracheal tube 12 can be inserted through the patient's nasal cavity N, and then advanced to the vocal folds VF. As with an orotracheal approach, the inflatable cuff 20 can be maintained in a deflated position to facilitate passage through the airway.

To provide confirmation that the endotracheal tube 12 has been inserted through the vocal folds VF, the first ultrasonic transducer 106 on the upper section 100 of the ultrasound apparatus 96 can be selectively activated, producing an ultrasonic wave can be transmitted into the body and reflected against the distal section 16 of the endotracheal tube 12. The movement of the endotracheal tube 12 within the airway as a result of the vibration mechanism 40 causes the incident ultrasonic wave pulse to undergo a phase shift as it is reflected back to the first ultrasonic transducer 106. This reflected ultrasound wave can then be sent to an ultrasound-imaging device that can be configured to produce an image on a screen using Doppler ultrasound techniques. Alternatively, the reflected ultrasonic waves can be sent to an auscultatory device configured to produce an audible tone that can be used to determine the precise location of the endotracheal tube 12 within the airway.

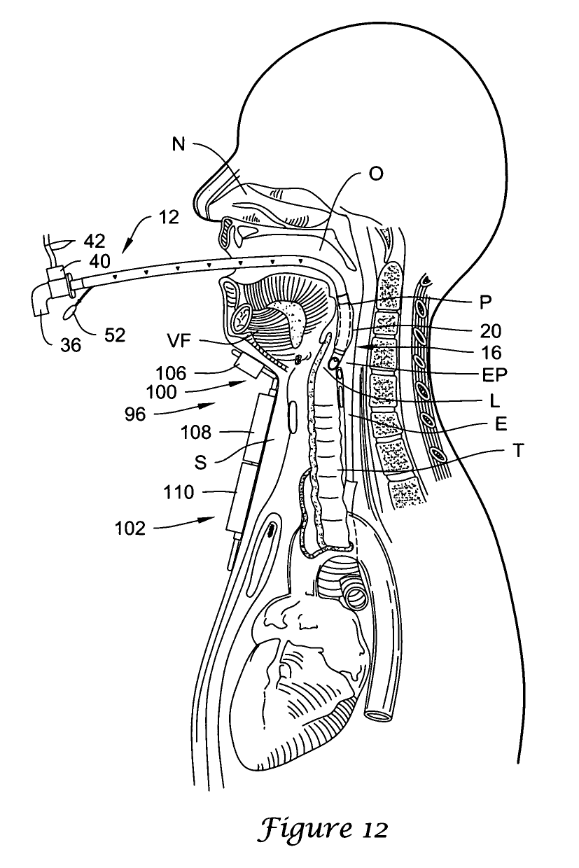

Once confirmation that the distal section 16 of the endotracheal tube 12 has been inserted and advanced to a position near the vocal folds VF, the caregiver next advances the endotracheal tube 12 to a second position within the body at or near the epiglottis EP and opening of the trachea T, as shown, for example, in FIG. 12. At this position, the second ultrasonic transducer 108 can also be activated to further visualize the endotracheal tube 12 using Doppler ultrasound techniques, allowing the caregiver to determine whether the endotracheal tube 12 is properly positioned along the anterior portion of the larynx/pharynx. The certain embodiments, the ultrasound apparatus 96 can be configured to provide an audible and/or visual alarm indicating that the endotracheal tube 12 has been improperly placed in the esophagus E or at some other undesired location, prompting the caregiver to reposition the endotracheal tube 12.

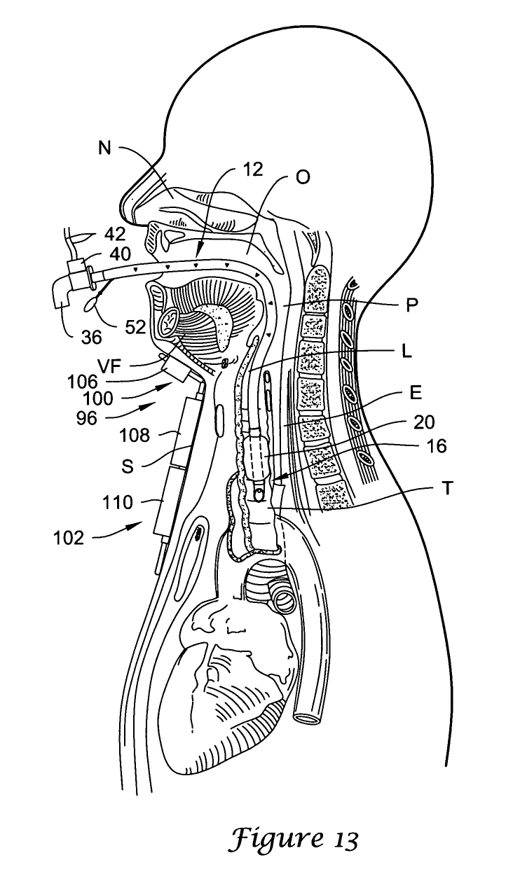

Once the caregiver has determined that the endotracheal tube 12 is properly positioned along the anterior portion of the larynx/pharynx at or near the epiglottis EP, the endotracheal tube 12 can then advanced into the trachea T guided by the location of the Doppler image resulting from the activation of the second and third ultrasonic transducers 108,110. Once tracheal intubation has been confirmed, the endotracheal tube 12 is then further advanced into the trachea T and secured therein by inflation of the inflatable cuff 20, as shown, for example, in FIG. 13.

To improve visualization of the endotracheal tube 12 within the body, the ultrasonic imaging apparatus can be configured to display only those frequencies associated with movement of the endotracheal tube 12. In certain embodiments, for example, the ultrasonic imaging apparatus can be configured to tune-out frequencies associated with blood flow, allowing only Doppler movement corresponding with vibration of the endotracheal tube 12 to be displayed.

Having thus described the several embodiments of the present invention, those of skill in the art will readily appreciate that other embodiments may be made and used which fall within the scope of the claims attached hereto. Numerous advantages of the invention covered by this document have been set forth in the foregoing description. It will be understood that this disclosure is, in many respects, only illustrative. Changes may be made in details, particularly in matters of shape, size and arrangement of parts without exceeding the scope of the invention as described in the appended claims.

1. (canceled)

2. A system for ultrasonically placing and monitoring an endotracheal tube within the airway of a patient, the system comprising:
an endotracheal tube having a proximal end, a distal end, and ventilation lumen disposed therethrough;
a vibration mechanism operatively coupled to the endotracheal tube;
at least one ultrasonic transducer located outside of the patient's body; and
ultrasonic imaging means for visualizing the endotracheal tube within the body.

3. The system of claim 1, further comprising a ventilation hub connected to the proximal end of the endotracheal tube, said ventilation hub in fluid communication with the ventilation lumen and an external ventilation unit.

4. The system of claim 1, wherein the endotracheal tube further includes an inflation lumen in fluid communication with an inflatable cuff.

5. The system of claim 1, wherein the vibration mechanism includes a piezoelectric actuator.

6. The system of claim 5, wherein said piezoelectric actuator is a Macro Fiber Piezocomposite (MFP) actuator.

7. The system of claim 1, wherein the vibration mechanism is coupled to a vibration hub.

8. The system of claim 7, wherein the vibration hub is secured to the proximal end of the endotracheal tube.

9. The system of claim 1, wherein the vibration mechanism is adapted to induce a transverse-mode vibration along the entire length of the endotracheal tube.

10. The system of claim 1, wherein said at least one ultrasonic transducer comprises a plurality of ultrasonic transducers.

11. The system of claim 10, wherein said plurality of ultrasonic transducers comprises an array of ultrasonic transducers.

12. The system of claim 1, wherein said ultrasonic imaging means comprises an apparatus adapted to ultrasonically image the endotracheal tube within the patient's airway using Doppler imaging.

13. A system for ultrasonically placing and monitoring an endotracheal tube within the airway of a patient, the system comprising:
an endotracheal tube having a proximal end, a distal end, and ventilation lumen disposed therethrough;
a vibration mechanism operatively coupled to the endotracheal tube;
an extracorporeal ultrasonic apparatus including a plurality of ultrasonic transducers, said ultrasonic apparatus including a mandible having an upper section adapted to lie adjacent to the anterior surface of the patient's neck, and a lower section adapted to lie adjacent to the anterior surface of the patient's sternum; and
ultrasonic imaging means for visualizing the endotracheal tube within the body.

14. The system of claim 13, further comprising a ventilation hub connected to the proximal end of the endotracheal tube, said ventilation hub in fluid communication with the ventilation lumen and an external ventilation unit.

15. The system of claim 13, wherein the endotracheal tube further includes an inflation lumen in fluid communication with an inflatable cuff.

16. The system of claim 13, wherein the vibration mechanism includes a piezoelectric actuator.

17. The system of claim 16, wherein said piezoelectric actuator is a Macro Fiber Piezocomposite (MFP) actuator.

18. The system of claim 13, wherein the vibration mechanism is coupled to a vibration hub.

19. The system of claim 18, wherein the vibration hub is secured to the proximal end of the endotracheal tube.

20. The system of claim 13, wherein the vibration mechanism is adapted to induce a transverse-mode vibration along the entire length of the endotracheal tube.

21. The system of claim 13, wherein said ultrasonic imaging means comprises an apparatus adapted to ultrasonically image the endotracheal tube within the patient's airway using Doppler imaging.

22. The system of claim 13, wherein said plurality of ultrasonic transducers comprises:
a first ultrasonic transducer disposed on the upper section of the mandible; and
a second ultrasonic transducer disposed on the lower section of the mandible.

23. The system of claim 22, wherein said first ultrasonic transducer comprises a plurality of ultrasonic transducer elements.

24. The system of claim 22, wherein said first ultrasonic transducer comprises an array of ultrasonic transducer elements.

25. The system of claim 22, wherein said second ultrasonic transducer comprises a plurality of ultrasonic transducer elements.

26. The system of claim 22, wherein said second ultrasonic transducer comprises an array of ultrasonic transducer elements.

27. The system of claim 13, wherein the mandible includes a bendable joint configured to permit movement of the upper section of the mandible relative to the lower section thereof.

28. The system of claim 13, wherein the upper section of the mandible has a concaved surface adapted to permit the mandible to least partially surround the anterior surface of the patient's neck and sternum when attached thereto.

29. A method of ultrasonically placing and monitoring an endotracheal tube within the airway of a patient, the method comprising the steps of:
providing an endotracheal tube having a proximal end, a distal end, and ventilation lumen disposed therethrough;
providing an ultrasonic apparatus including a plurality of ultrasonic transducers and attaching the ultrasonic apparatus to the anterior portion of the patient's neck and sternum;
inserting at least a portion of the endotracheal tube into the patient's oral or nasal cavity and advancing the tube into the patient's airway;
vibrating the endotracheal tube; and
ultrasonically visualizing the endotracheal tube within the airway.

30. The method of claim 29, wherein the endotracheal tube further includes an inflatable cuff in fluid communication with an inflation fluid source, and further comprising the step of expanding the inflatable cuff to provide an air seal within the airway.

31. The method of claim 29, further comprising the step of ventilating the patient using an external ventilating unit operatively coupled to the ventilation lumen.

32. The method of claim 29, wherein said step of ultrasonically visualizing the endotracheal tube within the airway comprises the steps of:
ultrasonically visualizing the endotracheal tube at a first position within the airway using a first ultrasonic transducer; and
ultrasonically visualizing the endotracheal tube at a second position within the airway using a second ultrasonic transducer.

33. The method of claim 32, wherein each of said first and second ultrasonic transducers comprises a plurality of ultrasonic transducers.

34. A method of ultrasonically placing an endotracheal tube within the airway of a patient, the method comprising the steps of:
providing an endotracheal tube having a proximal end, a distal end, and ventilation lumen disposed therethrough;
providing an ultrasonic apparatus including a mandible having plurality of ultrasonic transducers and attaching the mandible to the anterior portion of the patient's neck and sternum;
inserting at least a portion of the endotracheal tube into the patient's oral or nasal cavity and advancing the tube into the patient's airway;
vibrating the endotracheal tube;
selectively activating a first ultrasonic transducer on an upper section of the mandible and ultrasonically visualizing the endotracheal tube at a first position within the patient's airway;
advancing the endotracheal tube to a second position within the airway; and
selectively activating a second ultrasonic transducer on a lower section of the mandible and ultrasonically visualizing the endotracheal tube at a second position within the patient's airway.

35. The method of claim 34, wherein the endotracheal tube further includes an inflatable cuff in fluid communication with an inflation fluid source, and further comprising the step of expanding the inflatable cuff to provide an air seal within the airway.

36. The method of claim 34, further comprising the step of ventilating the patient using an external ventilating unit operatively coupled to the ventilation lumen.
